(19) United States
(12) Patent Application Publication
(10) Pub. No.: US 2004/0018584 A1
(43) Pub. Date: Jan. 29, 2004

(54) Method of detecting and estimating water contamination by aerobic spores

(76) Inventor: Per Einar Granum, Oslo (NO)

Correspondence Address: DUNLAP, CODDING & ROGERS P.C., PO BOX 16370, OKLAHOMA CITY, OK, 73113, US

(21) Appl. No.: 10/319,945

(22) Filed: Dec. 13, 2002

Related U.S. Application Data
(60) Provisional application No. 60/340,128, filed on Dec. 14, 2001.

(51) Int. Cl. C12Q 1/04

(52) U.S. Cl. 435/34

(57) ABSTRACT

The present invention is a system and method for early detection of contamination of water by aerobic spores based on fluorescence measurements of water samples incubated with growth medium. The method can detect major discrete contamination events by aerobic spores of bacteria such as Bacillus sp., including Bacillus cereus, B. anthracis (anthrax) and B. subtilis, within 2 to 10 hours. The method can be used in a lab-based system or as part of a remote warning system taken at a discharge point upstream from a user of the water source. The method can also be used as an indicator of the presence of Cryptosporidium parvum.

### CROSS REFERENCE TO RELATED APPLICATIONS

The present application claims benefit of U.S. Provisional application No. 60/340,128, filed Dec. 14, 2001, which is hereby expressly incorporated herein by reference in its entirety.

### STATEMENT REGARDING FEDERALLY SPONSORED RESEARCH OR DEVELOPMENT

Not applicable.

### BACKGROUND

The invention relates to methods of detecting water contamination, and more particularly, though not by limitation, to methods of detecting contamination by aerobic spores, including spores of Bacillus sp.

The terror attacks of Sep. 11, 2001 have caused an exhaustive review of the present security measures used in the water industry. Both chemical and biological sabotage are possible, with addition of agents to the water supply, treatment plants, or to community distribution systems. The likelihood of a successful attack, i.e. causing extensive illness and mortality, is not considered extreme. However, the long term effect has already resulted in revision of security measures and contingency plans.

In the short term, the security measures primarily include more diligent monitoring for pathogens. Long term security will necessitate investments in monitoring systems. Estimates by the Centers for Disease Control suggest that close to a million cases of illnesses and 900 deaths occur every year in the U.S. alone as a result of waterborne microbial infection. What the threat of bioterrorism has certainly achieved is to bring to the fore the issue of microbial water pollution that was otherwise eclipsed by chemical and other risks. Associated with the threat of bioterrorism to smaller and non-community water systems, the market for detection, monitoring and treatment for waterborne microorganisms is expected to increase. Water use in other areas such as air conditioning or food production are also candidate applications to benefit from early warning methods. It would be desirable to have methods which more rapidly detect the presence of spores in water systems than those which are currently available.

### BRIEF SUMMARY OF THE INVENTION

The present invention is a system and method for early detection of contamination of water by aerobic spores based on fluorescence measurements of water samples incubated with growth medium. The method can detect major discrete contamination events by aerobic spores of bacteria such as Bacillus sp., including Bacillus cereus, B. anthracis (anthrax) and B. subtilis, within 2 to 10 hours. The method can be used in a lab-based system or as part of a remote warning system taken at a discharge point upstream from a user of the water source. The method can also be used as an indicator of the presence of Cryptosporidium parvum.

### BRIEF DESCRIPTION OF THE DRAWINGS

FIG. 1. TTD curve for aerobic spores using Bacillus cereus reference culture strains. Incubation temperature: 37° C., Pre-Incubation Heat Treatment: 80° C., 10 minutes.

FIG. 2. TTD curve for aerobic spores using Bacillus cereus reference culture strains. Incubation temperature: 30° C., Pre-Incubation Heat Treatment: 80° C., 10 minutes.

FIG. 3. TTD curve for aerobic spores using Bacillus cereus reference culture strains and spiked with 1000 cfu E. coli per 1 ml. Incubation temperature: 37° C., Pre-Incubation Heat Treatment: 80° C., 10 minutes.

FIG. 4. TTD curve for aerobic spores using Bacillus cereus reference culture strains and spiked with Lysaker river water-(Vann). Incubation temperature: 37° C., Pre-Incubation Heat Treatment: 80° C., 10 minutes.

FIG. 5. TTD curve for aerobic spores using Bacillus cereus reference culture strains and Lysaker river water. Incubation temperature: 37° C. Pre-Incubation Heat Treatment: 80° C. for 15 minutes.

### DETAILED DESCRIPTION OF THE INVENTION

Early warning systems to provide improved security based on the automated detection of fluorescent products derived from target organism enzymatic activity have been developed by Colifast. Several indicators such as variants of the coliform group and total viable organisms are rapidly detected at low levels, using different combinations of organism enzymes and paired fluorogenic substrates. Presence-absence determinations are targeted to 50% of the reference method time-to-result (TTR). More importantly, however, is the aspect of detecting higher levels of target organisms, say 10 to 1000 cfu, which represent genuine contamination episodes that can require action. An “Early Warning” feature will automatically report such incidents in a few hours, the TTR being inversely proportional to the level of contamination. The two instruments that enable this capability are: the lab-based Automated Microbe Analyzer (AMA) and operations or “at-line” based Colifast At-line Monitor (CALM).

The present invention is a system and method for early detection of contamination of water by aerobic spores based on fluorescence measurements of water samples incubated with growth medium. The method can detect major discrete contamination events by aerobic spores of bacteria including Bacillus sp. such as Bacillus cereus, anthrax (Bacillis anthracis), and Bacillus subtilis in from approximately 2 to 10 hours. The method can be used in a lab-based system or as part of a remote warning system taken at a discharge point upstream from a user of the water source. For example, the water sample may be obtained at a point in an master source such as a river which is upstream (for example at least 10 hours upstream as measured by flow rate) of a water use or water treatment facility such as a municipal treatment facility or commercial or industrial use.

In general, the invention is a method for detecting and estimating contamination of water samples by aerobic bacterial spores. The method comprises providing a water sample, heating the water sample or a portion thereof at a predetermined heat treatment temperature for a predetermined duration of time wherein non-spore, vegetative stage bacteria are killed but the spores of aerobic bacteria are not killed, combining the heated water sample with a growth medium having a fluorogenic substrate to form an incubation mixture, incubating the incubation mixture at a predetermined incubation temperature, exposing at least a portion of the incubation mixture to an excitation wavelength after a predetermined incubation time and measuring the fluorescence emitted from the incubated incubation mixture to obtain a fluorescence measurement, and then making a determination of the spore concentration in the water sample based on the fluorescence measurement.

In one embodiment of the method, a plurality of fluorescence measurements based on subsamples of the water sample are obtained at predetermined time intervals until at least one of the fluorescence measurements exceeds a predetermined fluorescence threshold. The predetermined time interval is preferably about one hour. Preferably, the spore concentration is determined by comparing the fluorescence measurement to a predetermined correlation schedule. Aerobic spores are determined to be absent when the fluorescence threshold is not exceeded after ten hours of incubation. Furthermore, the predetermined correlation schedule may be based on a reference sample of aerobic spores having a known concentration of spores, wherein, for example, the reference sample comprises Bacillus spores, for example, Bacillus cereus spores.

Generally, in the step of heating the water sample, the heat treatment temperature is at least 80° C. and is less than 100° C., and preferably the heat treatment temperature is about 80° C. and the water sample is heated at the heat treatment temperature for about ten minutes to twelve minutes. The predetermined incubation temperature is from about 30° C. to about 37° C., and more preferably is about 37° C. ±0.5° C. In one embodiment of the invention, a spore concentration of 1,000,000 cfu/ml can be detected within about 2 hours, a spore concentration of 100,000 cfu/ml can be detected within about 3 to 4 hours, a spore concentration of 10,000 cfu/ml can be detected within about 4 to 5 hours, a spore concentration of 1,000 cfu/ml can be detected within about 4.5 to 6 hours, a spore concentration of 100 cfu/ml can be detected within about 5.5 to 7.5 hours, a spore concentration of 10 cfu/ml can be detected within about 6.5 to 7.5 to 8.5 hours, and a spore concentration of 1 cfu/ml can be detected within about 9 to 10 hours.

In an alternative embodiment, the method is carried out as part of a remote warning system wherein the water sample is obtained at a point in a water source (e.g., a river) at least ten hours upstream of a water treatment or water-use facility or plant.

### EXAMPLES

Methods

Preparation of Treatment Sample:

Prepare an 80° C. water-bath (shaking).

Add 100 μL of water sample which is desired to be tested for spores to 10 ml of dilution water to form a treatment sample.

Heat the treatment sample at 80° C. in water-bath for 10 minutes.

(In an alternative embodiment, the water sample to be tested may be heat treated before it is diluted by dilution water.)

Add 9 ml of the heated treatment sample to 1 ml Colifast TVO medium (or other fluorescence-type growth medium such as shown in U.S. Pat. No. 6,165,742, which is hereby expressly incorporated herein in its entirety) to form an incubation mixture.

Use Colifast Analyser (CA) for measurements once every hour (360 nm excitation wavelength; 485 emission wavelength is detected), or other fluorometric device such as a Turner Designs TD-700 fluorometer.

Preparation of High Calibration (CAL 480):

Add 9 ml of dilution water to 1 ml of Colifast TVO medium.

Add 500 μL of Colifast VHC (“Very High Calibration” solution; i.e., TVO medium plus a solution of methylumbelliferone (M [symbol] ) used for calibration).

Preparation of Low Calibration (Blank):

Add 9 ml of dilution water to 1 ml of Colifast TVO medium.

Materials:

Bacillus cereus reference culture (or other aerobic spore reference culture, e.g., Bacillus subtilis, ATCC 6633).

Lysaker River Water.

E. coli (Colifast reference culture or other E. coli reference culture).

Dilution water.

Colifast TVO medium.

Colifast VHC.

Water-bath 80° C. (Pre-treatment of the treatment sample at 80° C. for 10 minutes functions to kill actively growing, non-spore, vegetative-stage microorganisms thus making the test specific for spores).

Pipettes.

Timer.

Colifast Analyser (or other fluorometric device).

Optimised TVO-medium and Procedure for Preparing the Medium:

1.Solution A: Dissolve 2.83 g TVO-Basic-medium* (1 bottle) in 90 ml distilled water in a Duran bottle. Stir to dissolve. Autoclave at 121 (C for 15 min to sterilise. Allow cooling to room temperature.

2.Solution B: Add 5 ml acetone and 5 ml Dimethyl sulfoxide (DMSO) successively to one 115 mg-bottle of TVO-substrate-mix**. Mix well to dissolve.

3.Final TVO-medium: Add Solution B (10 ml) to Solution A (90 ml) by sterile filtration (0.2 (m pore size)***. Make sure that sterile technique is used. Stir for at least 10 min.

4.Precipitation may be observed in the final TVO-medium, and it is therefore recommended to fill media in vials immediately. Stir well to obtain a “homogeneous” solution and pipette 1 ml final TVO-medium to pre-autoclaved vials (totally 95-100 vials). Make sure that sterile technique is used. Vials containing final media may be stored in the dark at 4-8 (C for four weeks. Precipitation may be seen in the vials, but this will not reduce the performance of the test.

*TVO-basic medium: Bacto Yeast Extract (0.5 g), Bacto Proteose Peptone (0.5 g), Casamino Acids (0.5 g), Dextrose (0.59), Soluble Starch (0.2 g)#, Sodium Pyruvate (0.3 g), Potassium Phosphate(3H₂O (0.28 g)##, Magnesium Phosphate (0.05 g), distilled water (90 ml).

This medium is 10× concentrated R2A medium without agar with two additional modifications:

#Soluble Starch concentration is reduced from 0.5 g to 0.2 g (to reduce precipitation of starch in the liquid medium); and

##Potassium Phosphate(3H₂O is reduced from 0.3 g to 0.28 g (to obtain pH 7.0).

**TVO substrate mix: 4-methylumbelliferyl-(-D-glucoside (50 mg), 4-methylumbelliferyi-Phosphate (50 mg), 4-methylumbelliferyl-Palmitate (15 mg).

***The substrate solution or the final medium must not be autoclaved because the substrates will auto-hydrolyse at high temperatures. The substrate solution is therefore added to the basic medium by sterile filtration.

Experiment 1

Purpose: To obtain a time-to-detect (TTD) curve for aerobic spores using Bacillus cereus reference culture strains.

Method:

Prepare the treatment sample as described above in “Preparation of Treatment Sample”.

Make dilutions of the treatment sample after heat treatment.

Dilutions:

1 cfu per 1 ml of treatment sample.

10 cfu per 1 ml of treatment sample.

100 cfu per 1 ml of treatment sample.

1000 cfu per 1 ml of treatment sample.

10000 cfu per 1 ml of treatment sample.

Add 9 ml of sample dilution to 1 ml of a growth medium such as

Colifast TVO medium.

Incubate the incubation samples at 37° C. using the Colifast Analyser (or other fluorescence analyzer).

Use Colifast Analyser for measurements once every hour for 10 hours.

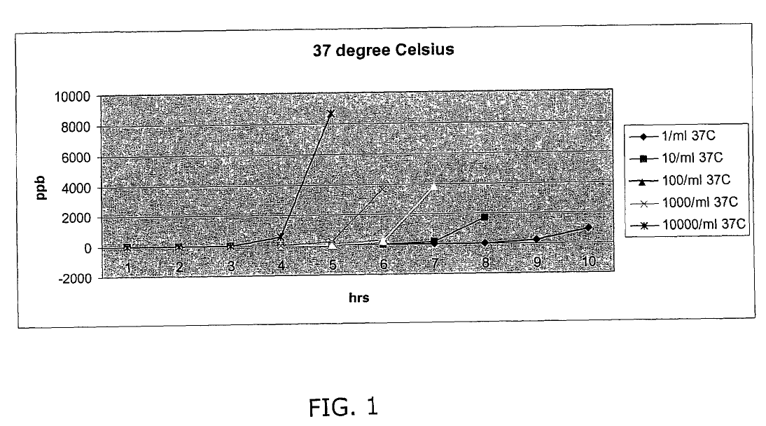

Results: see FIG. 1.

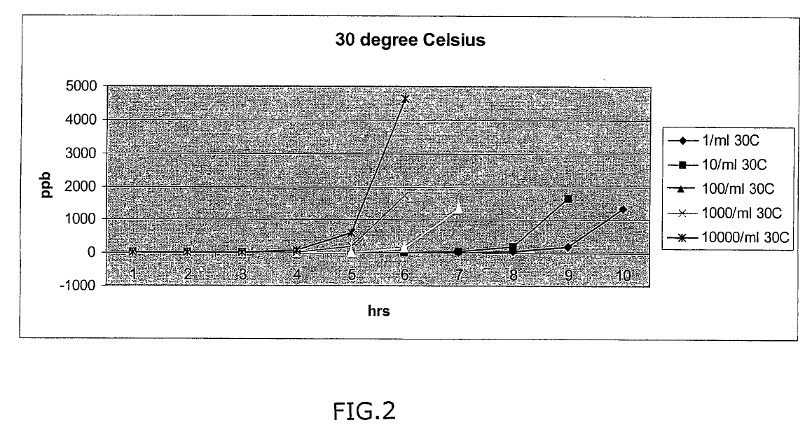

Another set of samples was incubated at 30° C. using the Colifast Analyser. The samples were prepared exactly as described above. Results: See FIG. 2.

Based on these results, 37° C. is a more optimal incubation temperature for aerobic spores from Bacillus cereus reference culture strains.

With incubation at 37° C., Bacillus cereus spores can be detected in about 4 hours when spore concentration is about 10,000 CFU/ml and in about 9-10 hours when spore concentration is 1 CFU/ml.

Experiment 2

Purpose: to challenge the method by adding 1000 cfu E. coli per 1 ml to one sample and Lysaker river water (vann) instead of dilution water to the next sample.

Method

Prepare the samples as described above in “Preparation of Treatment Sample”.

Add E. coli and Lysaker river water to the appropriate samples before heat treatment.

Make dilutions before heat treatment.

Dilutions:

10 cfu per 1 ml of treatment sample.

100 cfu per 1 ml of treatment sample.

1000 cfu per 1 ml of treatment sample.

10000 cfu per 1 ml of treatment sample.

Incubate the samples at 37° C. using the Colifast Analyser.

Use Colifast Analyser for measurements once every hour for 4-7 hours.

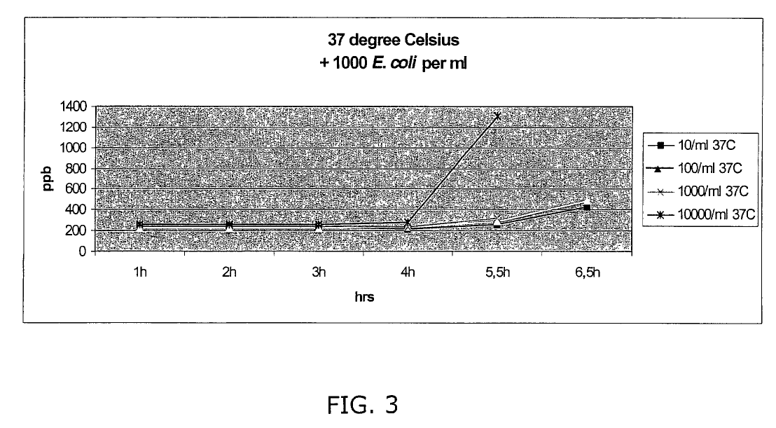

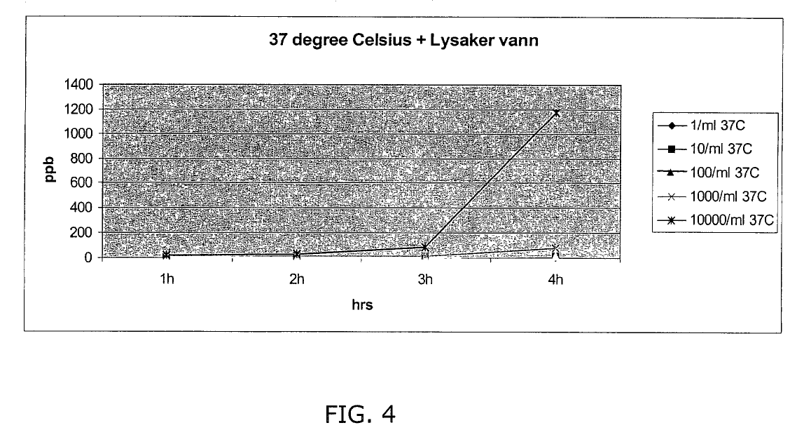

Results: see FIGS. 3 and 4. The results indicate that neither the addition of actively growing bacteria (E. coli) nor microorganisms in river water compromise the overall results of the method.

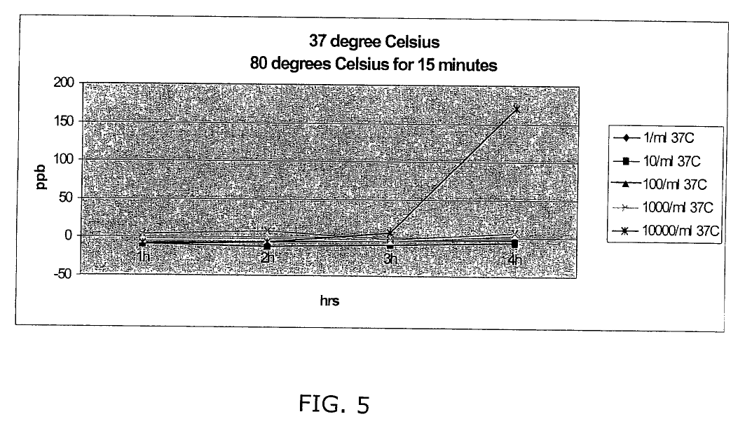

For another set of samples, the 80° C. heat treatment was extended from 10 minutes to 15 minutes. See FIG. 5. The test was performed exactly as described above. Heat treatment for 15 minutes appears to provide no advantage over 10 minutes.

As indicated herein a spore concentration in a water sample is estimated by first determining background fluorescence, and then determining a threshold fluorescence level which exceeds the background level at a desired safety level. When the threshold fluorescence level is surpassed after a period of incubation, it is concluded that aerobic spores are present in the original water sample. A correlation schedule (a time-to-detect curve) is formulated for each testing location, which relates time (minutes or hours of incubation) to spore concentration (cfu/ml) based on dilutions of a standard spore sample having a known concentration, or based on other standard reference methods, e.g., those used by the U.S. E.P.A. and known to those of ordinary skill in the art. When a fluorescence measurement from an incubated water sample which exceeds the predetermined threshold fluorescence level is obtained, the incubation time associated with that fluorescence measurement is compared to the correlation schedule to estimate the concentration of spores in the original water sample. If after 10 hours of incubation the fluorescence threshold level is not exceeded, the water sample is concluded as having less than 1 cfu/ml.

The water sample to be tested can be subdivided into a plurality of subsamples wherein each is incubated separately and a fluorescence measurement is made separately, or the initial water sample can be incubated, and fluorescence measurements of the initial water sample can be taken by removing repeated subsamples therefrom.

In another embodiment of the present invention, the heated water sample is incubated on agar plates (comprising the fluorogenic growth medium) to provide a colony count method, wherein the plates are incubated for 10 hours at 37° C., after which colonies are determined numerically via fluorescence excitation.

The present invention is not to be limited in scope by the specific embodiments described, herein, since such embodiments are intended as bu single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

What is claimed is:

1. A method for detecting and estimating contamination of water samples by aerobic bacterial spores; comprising:
providing a water sample;
heating the water sample or a portion thereof at a heat treatment temperature and for a duration of time which kills non-spore, vegetative stage bacteria but which does not kill the spores of aerobic bacteria;
combining the heated water sample with a growth medium having a fluorogenic substrate to form an incubation mixture;
incubating the incubation mixture at a predetermined incubation temperature;
exposing at least a portion of the incubation mixture to an excitation wavelength after a predetermined incubation time and measuring the fluorescence emitted from the incubation mixture to obtain a fluorescence measurement; and
making a determination of the spore concentration in the water sample based on the fluorescence measurement.

2. The method of claim 1 wherein a plurality of fluorescence measurements based on subsamples of the water sample are obtained at predetermined time intervals until at least one of the fluorescence measurements exceeds a predetermined fluorescence threshold.

3. The method of claim 2 wherein the predetermined time interval is about one hour.

4. The method of claim 1 wherein the method detects total aerobic bacterial spores.

5. The method of claim 1 wherein in the step of making a determination of the spore concentration, the spore concentration is determined by comparing the fluorescence measurement to a predetermined correlation schedule, or wherein aerobic spores are determined to be absent in the water sample if the fluorescence threshold is not exceeded after ten hours of incubation.

6. The method of claim 5 wherein the predetermined correlation schedule is based on a reference sample of aerobic spores having a known concentration of spores, or is based on a standard reference method.

7. The method of claim 6 wherein the reference sample comprises Bacillus spores.

8. The method of claim 7 wherein the reference sample comprises Bacillus cereus spores.

9. The method of claim 1 wherein in the step of heating the water sample, the heat treatment temperature is at least 80° C. and is less than 100° C.

10. The method of claim 1 wherein in the step of heating the water sample, the heat treatment temperature is about 80° C. and the water sample is heated at the heat treatment temperature for about ten minutes to twelve minutes.

11. The method of claim 1 wherein the predetermined incubation temperature is from about 30° C. to about 37° C.

12. The method of claim 1 wherein the predetermined incubation temperature is about 37° C.±0.5° C.

13. The method of claim 1 wherein a spore concentration of 1,000,000 cfu/ml can be detected within about 2 hours.

14. The method of claim 1 wherein a spore concentration of 100,000 cfu/ml can be detected within about 3 to 4 hours.

15. The method of claim 1 wherein a spore concentration of 10,000 cfu/ml can be detected within about 4 to 5 hours.

16. The method of claim 1 wherein a spore concentration of 1,000 cfu/ml can be detected within about 4.5 to 6 hours.

17. The method of claim 1 wherein a spore concentration of 100 cfu/ml can be detected within about 5.5 to 7.5 hours.

18. The method of claim 1 wherein a spore concentration of 10 cfu/ml can be detected within about 6.5 to 8.5 hours.

19. The method of claim 1 wherein a spore concentration of 1 cfu/ml can be detected within about 9 to 10 hours.

20. The method of claim 1 wherein the method is carried out as part of a remote warning system wherein the water sample is obtained at a point in a water source at least ten hours upstream of a water treatment or water-use facility or plant.

21. The method of claim 20 wherein the water source is a river.
